(19) United States
(12) Patent Application Publication
(10) Pub. No.: US 2025/0302856 A1
(43) Pub. Date: Oct. 2, 2025

(54) METHODS AND NUTRACEUTICAL COMPOSITIONS FOR THE PROMOTION OF WAKEFULNESS AND ALERTNESS

(71) Applicant: Kaplan IP, LLC, Hallandale Beach, FL (US)

(72) Inventors: Linda J. Kaplan, Hallandale Beach, FL (US); Rachel K. Grossman, Hallandale Beach, FL (US)

(73) Assignee: Kaplan IP, LLC, Hallandale Beach, FL (US)

(21) Appl. No.: 18/621,794

(22) Filed: Mar. 29, 2024

(51) Int. Cl. A61K 31/7068; A61K 31/19; A61K 31/197; A61K 31/198; A61K 31/375; A61K 31/4415; A61K 31/455; A61K 31/51; A61K 31/522; A61K 31/525; A61K 31/714; A61P 25/26

(52) U.S. Cl. CPC A61K 31/7068; A61K 31/19; A61K 31/197; A61K 31/198; A61K 31/375; A61K 31/4415; A61K 31/455; A61K 31/51; A61K 31/522; A61K 31/525; A61K 31/714; A61P 25/26

(57) ABSTRACT

Nutraceutical compositions and the methods for promoting wakefulness and alertness by the administration thereof. The nutraceutical compositions address the challenged state of the individual to remain awake and alert during waking hours. The methods provide the nutraceutical composition directly to the cells in the brain and body where energy is produced and utilized. A nutraceutical composition includes a therapeutically effective amount of Vitamin B1, Vitamin B2, Vitamin B3, Vitamin B5, Vitamin B6, Vitamin B12, citicoline sodium, calcium pyruvate, and L-tyrosine, wherein said therapeutically effective amount promotes wakefulness by providing molecular support for glycolysis, Krebs cycle reactions, and electron transport chain (ETC) activity. The composition can further include Vitamin C and caffeine.

### BACKGROUND

Under ideal circumstances we awaken after a restful sleep, invigorated and alert. For those who do not awaken in an invigorated and alert state, breakfast and/or caffeine, often in the form of coffee or tea, is employed to provide a jolt or boost of energy. As the day passes and we become hungry, many of us ingest lunch. Unfortunately, depending on what and how much we eat, we may find ourselves in a postprandial slump.

Lack of energy during the solar day negatively impacts our ability to stay awake, be alert, focus, concentrate, be attentive, respond briskly, quickly and or actively, be effective and dynamic, feel vital, buoyant, excited, peppy, snappy, or sprightly, fulfill our responsibilities, and results in overall diminished quality of life, efficiency, effectiveness, and causes drowsiness.

When faced with a lack of wakefulness and/or alertness (hereinafter W/A), people have employed several techniques, including reducing air temperature, slapping their faces, increasing light brightness, playing loud energetic music, and using sniffing salts. These are short-lived solutions because we become acclimatized: the sting wears off, we have the ability to tune out sight and sound, and odors are transient.

When faced with a lack of W/A, people have also employed several consumable remedies such as coffee, tea, soda, energy drinks and other energy-related products. This prior art, which is primarily reliant on caffeine and often contains significant calories and sugar, may provide an immediate burst but is not long-lasting or sustainable. Furthermore, the prior art often results in jitters followed by an energy crash, which are not effects that individuals seek. While some of the prior art includes components that assist with elements of energy production and utilization, none plug into all three processes of energy production or the bridges between them, as this invention does.

The inventors recognized that there is a need to resolve the problems associated with prior art's efficacy and unwanted side effects.

Those skilled in the art could not have recognized that a composition such as the invention could be tenable, effective, pleasant, or possible.

### SUMMARY

Accordingly, it is an object of the invention to provide novel methods and a novel nutraceutical composition to increase W/A. Certain exemplary embodiments of the invention address the full scope of energy production and utilization by plugging into Glycolysis (aka Embden-Meyerhof-Parnas pathway (hereinafter EMP)), Krebs Cycle (hereinafter Krebs), Electron Transport Chain (ETC) and the bridges between them. Certain exemplary embodiments of the invention focus on increasing the Orexin system's (hereinafter OREX) impact on W/A either by increasing its production, decreasing its deterioration, or by increasing its efficacy.

By providing support to molecules in the brain and body, with the utilization of alternative delivery systems, the invention is uniquely effective in promoting W/A.

### BRIEF DESCRIPTION OF THE FIGURES

Advantages of embodiments of the invention will be apparent from the following detailed description of the exemplary embodiments. The following detailed description should be considered in conjunction with the accompanying figures in which:

FIG. 1A, in conjunction with FIGS. 1B-1C, represents an overview of aerobic respiration, including EMP (FIG. 1A), Krebs (FIG. 1B), and ETC (FIG. 1C).

FIG. 1B represents the pyruvate generated in FIG. 1A being converted to Acetyl COA, which enters Krebs.

FIG. 1C represents that electrons from NADH and FADH2 are transferred to ETC where more ATP is generated.

FIG. 2A, to be viewed in conjunction with FIG. 2B together representing that adenosine is phosphorylated to be ATP, shows a reaction in which adenosine, a purine nucleoside, has its phosphoanhydride bonds to phosphate restored, becoming energized ATP (FIG. 2B).

FIG. 2B represents energized ATP.

FIG. 3A, in conjunction with FIG. 3B, represents glycolysis (EMP), the cytoplasmic process of converting one molecule of glucose into two (2) pyruvates while netting two (2) ATP and two (2) NADH molecules.

FIG. 3B, continuing from FIG. 3A, shows that four (4) ATP are produced but two (2) are used up in the EMP process. Circulating glucose crosses the blood brain barrier (BBB) and is transported into neurons and glial cells.

FIG. 4A in conjunction with FIG. 4B-4C represents the Krebs cycle (Krebs), an aerobic respiratory process.

FIG. 4B continuing from FIG. 4A shows that when Acetyl COA enters mitochondrial matrix, Krebs takes place.

FIG. 4C continuing from FIG. 4B, shows the subsequent stage of Krebs represented in FIGS. 4A-4C.

FIG. 5 illustrates the electron transport chain (ETC), the process of converting NADH and FADH2 into ATP.

FIG. 6 shows that the VLPO nucleus, located in the hypothalamus, acts like a toggle switch between W/A and sleep.

FIG. 7A shows molecular structure diagrams for adenosine, which is a purine nucleotide, and caffeine, which is a xanthine purine.

FIG. 7B shows that caffeine is an antagonist to adenosine receptors by binding to adenosine receptors in the brain, thereby blocking adenosine from binding.

FIG. 8 is a biology process diagram showing oxygenated nutraceutical-enriched blood (ONE) distribution via direct absorption into bloodstream.

FIG. 9 is a biology process diagram showing ONE distribution via ingestion.

### DETAILED DESCRIPTION

Aspects of the invention are disclosed in the following description and related figures directed to specific embodiments of the invention. Those skilled in the art will recognize that alternate embodiments may be devised without departing from the spirit or the scope of the claims. Additionally, well-known elements of exemplary embodiments of the invention will not be described in detail or will be omitted so as not to obscure the relevant details of the invention. Further, to facilitate an understanding of the description, discussion of several terms used herein follows.

As used herein, the word “exemplary” means “serving as an example, instance or illustration.” The embodiments described herein are not limiting, but rather are exemplary only. It should be understood that the described embodiments are not necessarily to be construed as preferred or advantageous over other embodiments. Moreover, the terms “embodiments of the invention”, “embodiments” or “invention” do not require that all embodiments of the invention include the discussed feature, advantage or more of operation.

The human state of W/A is maintained by a complex web of many brain pathways and their respective components impacted by Circadian Rhythm, environmental factors, hormones and metabolism. The brain requires oxygen to be viable and neurotransmit. As we respire (breath), deoxygenated air of 16% oxygen (O2) and 4.4% carbon dioxide (CO2) is exhaled and replaced with inhaled fresh air containing 21% O2 and 0.04% CO2. This process allows red blood cells (RBCs) to transport O2 to tissues requiring it for life processes and to remove CO2 as waste product. This patent addresses two critical factors that impact the W/A web: Adenosine 5′-triphosphate (ATP) the “Energy Molecule” and OREX.

The human body is a complex organism made of 37.2 trillion cells, all using ATP in every physiologic process to maintain proper functioning. ATP reduces fatigue, increases strength and power, and improves body composition. As ATP becomes exhausted from use, it impairs the brain's capacity for W/A. When ATP is “used up”, it devolves into adenosine. Adenosine is created when adenine (a nitrogen-based substance) and ribose (a sugar) combine. As adenosine builds up in the bloodstream and organs, it interacts with specific cell receptors, inhibiting neural activity and causing drowsiness. When the body runs out of fuel in the form of easily digestible sugars from the food we eat, adenosine signals the body to become drowsy. This invention focuses on increasing ATP production.

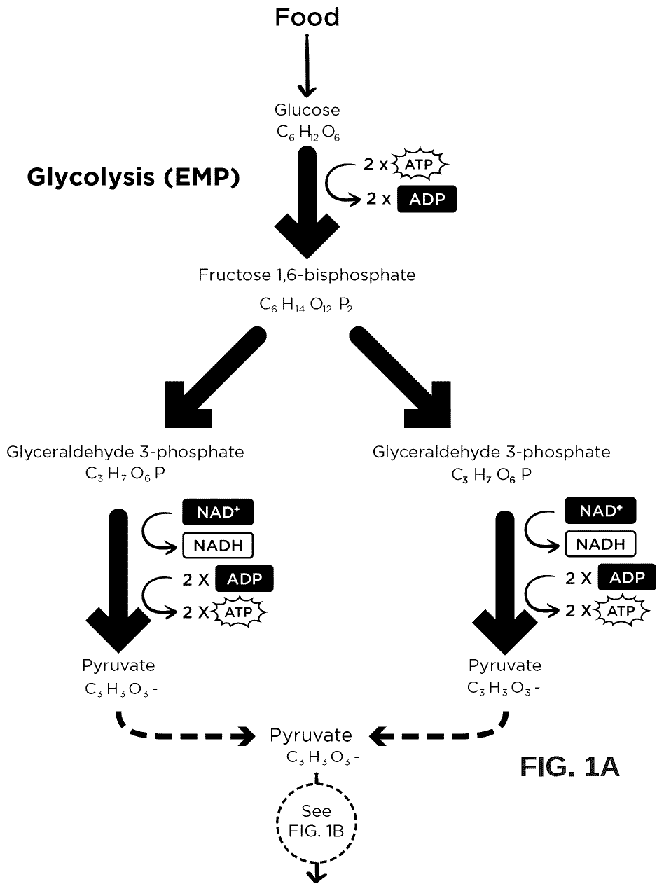

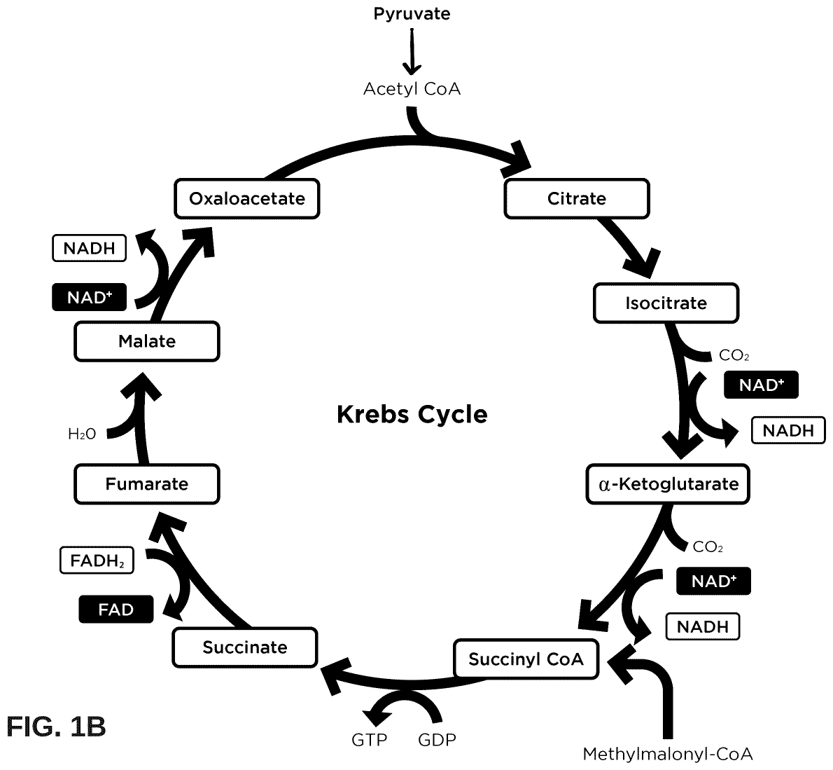

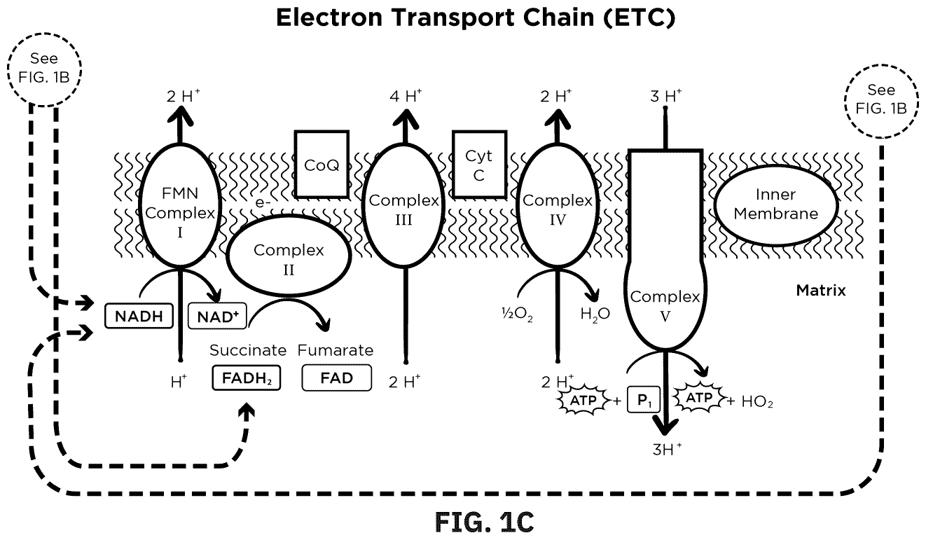

FIG. 1A-1C together provide an overview of aerobic respiration, including EMP (FIG. 1A), Krebs (FIG. 1B), and ETC (FIG. 1C). In humans, ATP is created through three processes: EMP, Krebs, and ETC. During EMP, glucose is converted to pyruvate, which is converted to Acetyl COA, which enters Krebs. Krebs (FIG. 1B) generates nicotinamide adenine dinucleotide and hydrogen (hereinafter NADH) and flavin adenine dinucleotide (FADH2). The electrons from NADH and FADH2 are transferred to ETC where more ATP is generated (FIG. 1C).

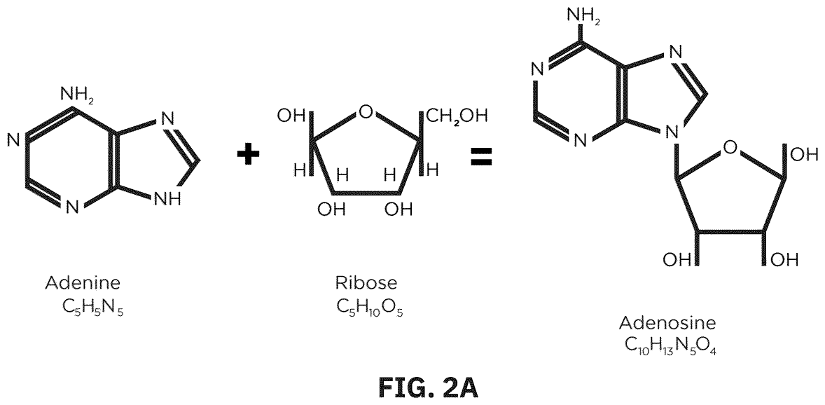

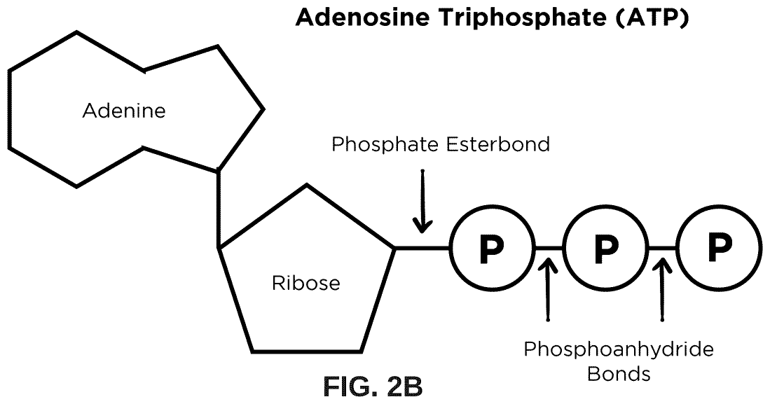

With reference to FIGS. 2A-2B, as adenosine, a purine nucleoside, has its phosphoanhydride bonds to phosphate restored, it becomes energized ATP (FIG. 2B). Certain embodiments of the invention work by providing the building blocks needed to produce and replenish ATP and to make ATP more readily available within each of these three processes. A difference between these processes is the net yield of ATP. Up to thirty-eight (38) ATP molecules can be made per oxidized glucose molecule during cellular respiration: two (2) from EMP, two (2) from Krebs, and about thirty four (34) from ETC.

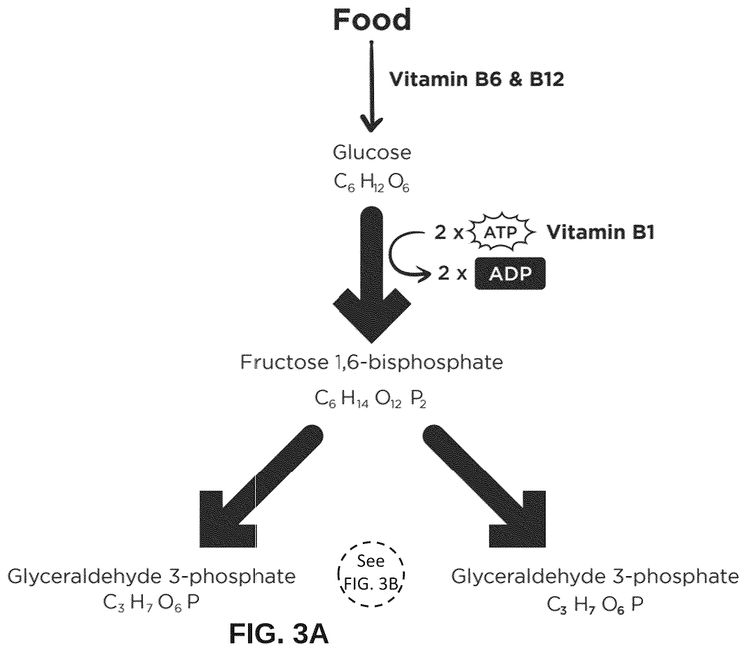

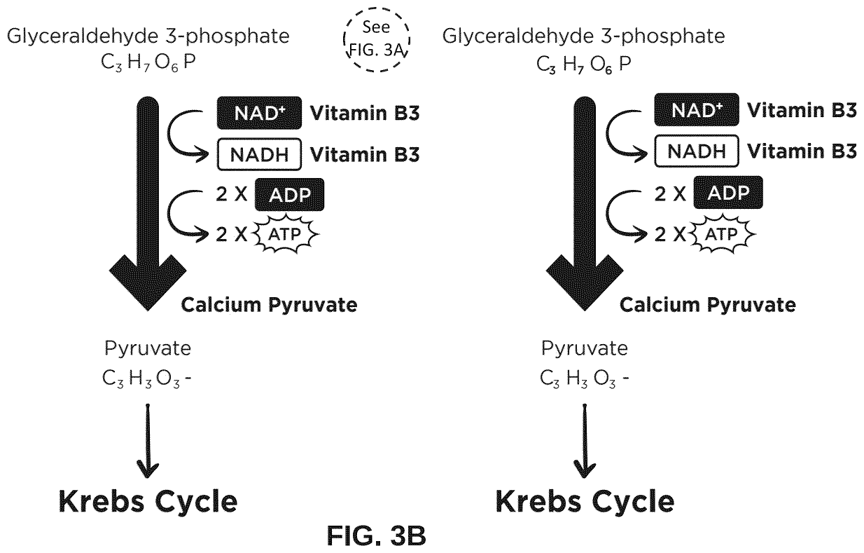

With reference to FIGS. 3A-3B, EMP is the cytoplasmic process of converting one molecule of glucose into two (2) pyruvates while netting two (2) ATP and two (2) NADH molecules. Note: four (4) ATP are produced but two (2) are used up in the EMP process. Circulating glucose crosses the blood brain barrier (BBB) and is transported into neurons and glial cells. The rate of EMP is impacted by Vitamins B1, B3, B6, and citicoline, and certain embodiments of the invention include Vitamins B1, B3, B6, Citicoline.

Vitamin B1 (B1), a thiamin complex, is comprised of thiamin pyrophosphate (TPP) and thiamin diphosphate (TDP). It impacts glucose metabolism and promotes the formation of more ATP via aerobic respiration. In glycolysis, it participates in the conversion of Pyruvate to Acetyl-CoA. TDP is a coenzyme upon which pyruvate dehydrogenase is dependent. Also, TPP is required in the pentose phosphate pathway which generates NADPH (Nicotinamide adenine dinucleotide phosphate) an essential electron donor that provides reducing power for anabolic reactions and redox balance which, in turn, keeps glutathione reduced to help mitigate oxidative damage. It also participates in Krebs. Certain embodiments of the invention include vitamin B1.

Vitamin B3 (B3), is comprised of Niacin, the generic name for nicotinic acid (pyridine-3-carboxylic acid), nicotinamide (niacinamide or pyridine-3-carboxamide), and related derivatives, such as nicotinamide riboside. It is involved in glucose metabolism and impacts glycolysis by participating in the conversion of Pyruvate to Acetyl-CoA (hereinafter Coenzyme A), and impacts Krebs. B3 is biosynthetically converted to nicotinamide adenine dinucleotide (NAD+), a versatile acceptor of hydride equivalents to form the reduced dinucleotide, NADH (NAD+ and hydrogen (H)). Essentially, NADH, is formed from B3, ribose, and adenosine diphosphate (ADP). NADH enters the final stage of cellular respiration and provides necessary electrons via ETC needed to power the creation of ATP. NAD and nicotinamide adenine dinucleotide phosphate (NADP), a homolog to NAD, are energy production coenzymes. NAD+ is the biologically functional form of NAD (nicotinamide adenine dinucleotide) containing catalytic properties. Certain embodiments of the invention include Vitamin B3.

Vitamin B6 (B6), an essential nutrient, is a complex of Pyridoxal 5′ phosphate (PLP or P5P), pyridoxamine 5′ phosphate (PMP), and pyridoxine. B6 is a coenzyme and cofactor for approximately 150 reactions that regulate the metabolism of glucose, lipids, amino acids, DNA, and neurotransmitters. It is involved in glycolysis and ETC and is integral to enzymes needed for the biosynthesis of neurotransmitters adrenaline (adrenalin), dopamine (dihydroxyphenethylamine), serotonin (5-hydroxytryptamine) and gamma-aminobutyric acid (GABA). Vitamin B6 also plays a role in gluconeogenesis. Gluconeogenesis refers to synthesis of new glucose from noncarbohydrate precursors, such as lactate, amino acids, and glycerol. In addition, it plays the role of antioxidant by counteracting the formation of reactive oxygen species (ROS) and advanced glycation end-products. Certain embodiments of the invention include Vitamin B6.

Acetylcholine (ACH) was the first molecule to be identified as a neurotransmitter important for W/A. It is also a neuromodulator. Its production is limited by the amount of available choline. Citicoline sodium once absorbed, citicoline (cytidine 5′-diphosphocholine), a nucleotide composed of cytosine, choline, ribose, and pyrophosphate crosses the BBB into the Central Nervous System (CNS). Citicoline provides the brain with choline that readily becomes ACH. Choline is transported into presynaptic neuron cells where it combines with acetyl CoA to synthesize ACH. Citicoline is a donor of choline in biosynthesis of choline-containing phosphoglycerides, and citicoline protects the liver by reducing oxidative stress in ETC. Certain embodiments of the invention include citicoline.

When intracellular glucose becomes phosphorylated by hexokinase, it becomes trapped inside the cell cytoplasm in the form of glucose 6 phosphate (G6P). In the brain, glucose phosphorylation is the rate limiting step in the process of glucose uptake and is necessary for neurotransmission. Hexokinase is comprised of glycine which can be synthesized from choline. Citicoline influences acetylcholine, dopamine, and glutamate neurotransmitter systems. Certain embodiments of the invention include citicoline.

The last catalyzing step of EMP cannot occur without the enzyme Pyruvate Kinase, and the first step of Krebs cannot occur without Pyruvate. After Pyruvate (the simplest keto acid) has been formed, it diffuses into mitochondria. In mitochondria, acetyl-CoA can be generated from the oxidation of Pyruvate, through fatty acid oxidation, by the degradation of the amino acids leucine, isoleucine, and tryptophan or through the mitochondrial enzyme acetyl-CoA synthetase. It generates reducing equivalents in the form of NADH and FADH2. Certain embodiments of the invention include Calcium Pyruvate.

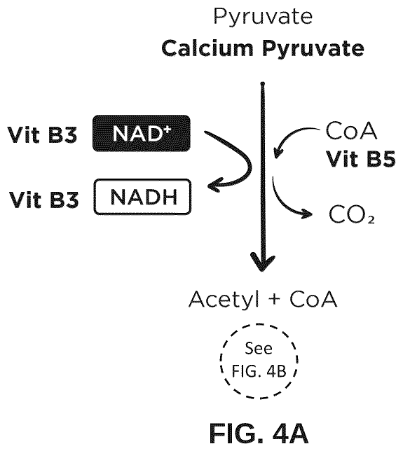

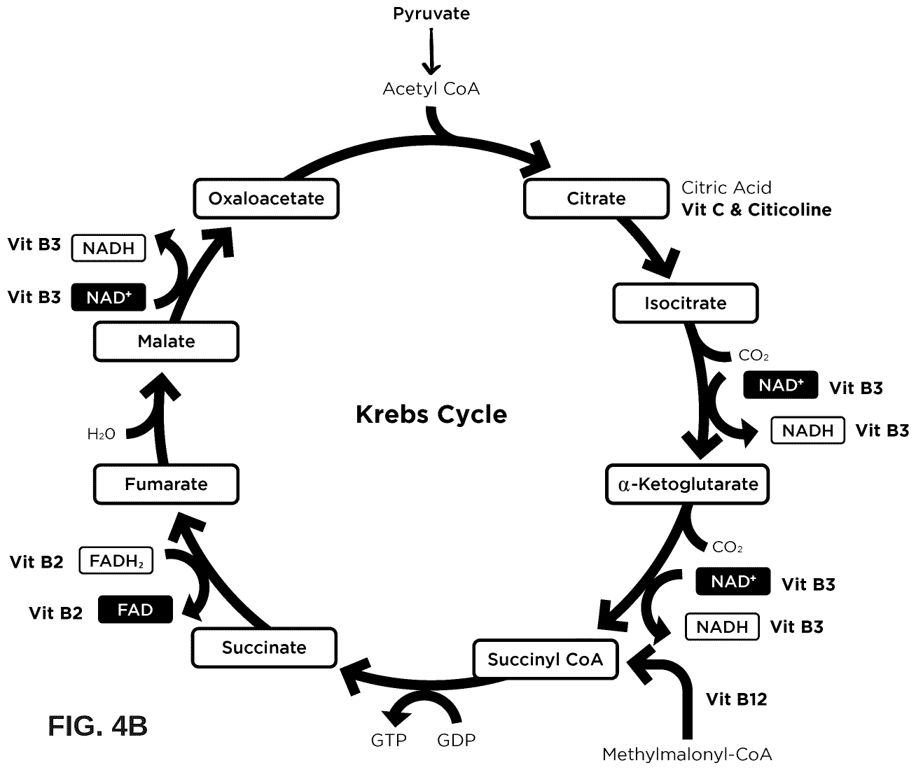

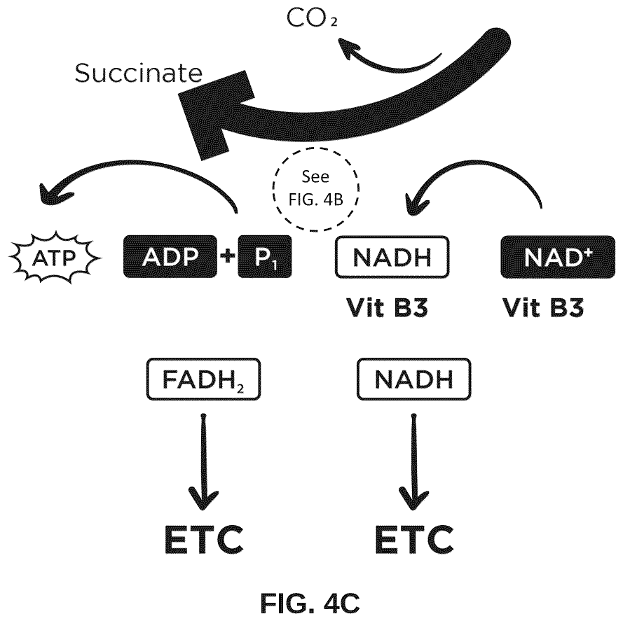

With reference to FIGS. 4A-4C, Krebs is an aerobic respiratory process that converts acetic acid or related substances such as Acetyl COA (a thioester between the acyl group carrier, acetic acid and a thiol, coenzyme A) into ATP. When Acetyl COA enters mitochondrial matrix, Krebs takes place. Oxidation occurs and ADP is converted into energy-rich ATP, leaving carbon dioxide (CO2) and water (H2O) as waste products. Krebs produces two (2) CO2, three (3) NADH, one (1) FADH2 and one (1) ATP. Krebs is supported by Vitamin B1, Vitamin B2 (B2), B3, Vitamin B5 (B5), B6, Vitamin B12 (B12), Vitamin C, and Calcium Pyruvate. Certain embodiments of the invention include VitaminB1, B2, B3, B5, B6, B12, Vitamin C and Calcium Pyruvate.

In Krebs, B1 participates as TPP, an important coenzyme for decarboxylation of pyruvate and oxidation of alpha-ketoglutamic acid. Certain embodiments of the invention include VitaminB1.

B2, aka riboflavin, helps EMP flow into Krebs as it participates in the conversion of Pyruvate to Acetyl-CoA. B2 serves as the building block for its co-enzymatic forms, flavin adenine dinucleotide (FAD) and flavin mononucleotide (FMN), which serve as electron carriers in various redox reactions in energy production and metabolic pathways. B2 transfers oxygen, which is required for cellular respiration, and as a vital component of ETC. Certain embodiments of the invention include VitaminB2.

In Krebs, B3 facilitates coenzymes NAD and NADP. Certain embodiments of the invention include VitaminB3.

B5 is the precursor to Coenzyme A (CoA). In EMP it is phosphorylated by ATP to form CoA to then enter Krebs. CoA is synthesized in a five-step process that requires four (4) molecules of ATP, pantothenate and cysteine. Certain embodiments of the invention include Vitamin B5.

B12 is a redox-active cofactor preventing cell death in the cytosol and mitochondria acting as an ROS scavenger and also participates in Krebs when it is bound to enzyme methylmalonyl-CoA mutase (MMCM) to mediate the isomerisation of methylmalonyl CoA to succinyl CoA. Certain embodiments of the invention include VitaminB12.

Vitamin C is an essential nutrient that activates pyruvate dehydrogenase (PDH) and also synthesizes L-carnitine in the liver which transports long-chain acyl groups from fatty acids into the mitochondrial matrix, so they can be oxidated to acetate to facilitate the citric acid cycle. Certain embodiments of the invention include Vitamin C.

In Krebs, as Pyruvate is decarboxylated and acetate is oxidized, electron carriers NADH and FADH are produced. These enter ETC.

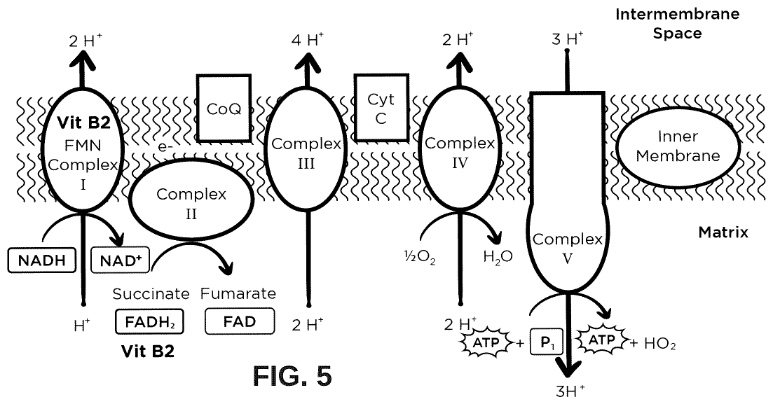

With reference to FIG. 5, ETC is the process of converting NADH and FADH2 into ATP. Oxygen, the final electron acceptor in ETC, is an excellent electron acceptor for the chemical reactions involved in generating ATP. ETC with Chemiosmosis is part of Oxidative Phosphorylation that nets thirty-four (34) ATP and one (1) molecule of H2O when electrons pass from carrier to carrier through a series of oxidation-reduction reactions. Oxidative phosphorylation differs from Krebs when H+ protons (hydrogen ions) are added by chemiosmosis. Oxidative phosphorylation consists of five (5) protein-lipid enzyme complexes located in the mitochondrial inner membrane that contain flavins FMN and FAD, quinoid compounds coenzyme Q10 (CoQ10), and transition metal compounds. Complex I accepts electrons from NADH and serves as the link between EMP, Krebs and ETC. Complex 2 serves as a link between Krebs and ETC. Complex 3 transfers electrons from CoQH2 to reduce cytochrome C. Complex 4 Cytochrome C is the substrate from which electrons are transferred to reduce oxygen into H2O. Complex 5 synthesizes ATP & H2O. The rate of ETC is impacted by B2 and B3. Certain embodiments of the invention include Vitamins B2 and B3. FIG. 5.

B2 serves as the building block for its co-enzymatic forms, flavin adenine dinucleotide (FAD) and flavin mononucleotide (FMN), which serve as electron carriers in various redox reactions in energy production and metabolic pathways to accept H atoms. B2 transfers oxygen, which is required for cellular respiration, and as a vital component of ETC. Certain embodiments of the invention include VitaminB2.

B3 Formed from B3, and ribose (a simple sugar), and adenosine diphosphate (ADP) is NADH. NADH enters the final stage of cellular respiration and provides necessary electrons via ETC needed to power the creation of ATP. NAD and nicotinamide adenine dinucleotide phosphate (NADP), a homolog to NAD, are energy production coenzymes. NAD+ is the biologically functional form of NAD (nicotinamide adenine dinucleotide) containing catalytic properties. Certain embodiments of the invention include VitaminB3.

An overview of the human nervous system formed of the central nervous system (CNS) and peripheral nervous system (PNS) will assist in understanding the OREX our invention promotes. The CNS consists of the brain and spinal cord. The peripheral nervous system (PNS) is divided into somatic and visceral systems, each with a sensory and a motor division. The visceral motor division aka the Autonomic Nervous System (ANS) regulates involuntary body functions.

The ANS has 2 divisions. The Sympathetic system that controls fight or flight stress responses; and the Parasympathetic system that controls rest and digest responses. The primary neurotransmitters of the Sympathetic system are adrenalin (aka epinephrine) a catecholamine neurotransmitter and hormone, and noradrenalin (aka norepinephrine) a catecholamine neurotransmitter and hormone. The adrenal glands produce adrenalin, noradrenalin and cortisol. The Parasympathetic system primarily utilizes ACH as a neurotransmitter. Vitamins B1, B5, and B12 affect adrenal gland cortisol activity which ties into energy production because cortisol triggers the release of glucose (sugar) into the blood (BS) from the liver for fast energy during times of stress (fight or flight response). L-Tyrosine impacts liver cells that utilize tyrosine hydroxylase for biosynthesis of adrenalin from noradrenalin. These systems have interplay because in the liver, Vitamin C helps biosynthesize carnitine which contributes to the synthesis of ACH. The adrenal glands also need Vitamin C to produce adrenalin and noradrenalin.

The PNS carries messages to and from the CNS. It is how the brain receives sensory information and controls all muscles.

The Ascending Arousal Network (AAN) aka RAS (reticular activating system) is activated by proprioception of joints through the spinocerebellar tract (i.e. you become aroused when someone shakes you); pain sensation (ex: aroused from skin prick through the spinothalamic and spinotectaltracts); sound (ex: a loud noise through the auditory system can jar you to attention); as well as visceral sensations via the General Visceral Afferent fibers (GVA).

The AAN is a subcortical neural network critical to consciousness. It connects to the brainstem, thalamus, hypothalamus, basal forebrain (BF) and cortex to activate W/A networks using glutamate, dopamine, noradrenaline, and ACH in conjunction with OREX.

OREX is produced in the hypothalamus by about 10,000-80,000 cells which is very few considering there are billions of brain cells. OREXs are neuropeptides and neurohormones. They have roles in regulating appetite, metabolism, the endocrine system, thermoregulation, reward systems, spontaneous physical activity, cardiovascular responses, and the sleep-wake cycle. They also promote arousal and are necessary for normal W/A.

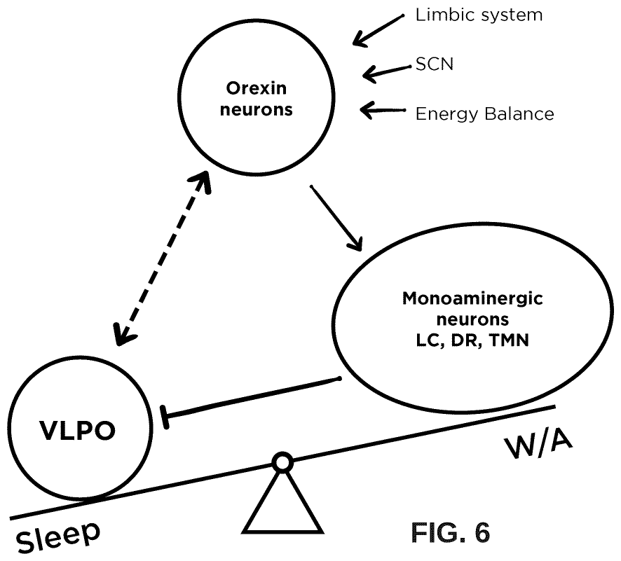

With reference to FIG. 6, located in the hypothalamus, the VLPO (ventrolateral preoptic) nucleus acts like a toggle switch between W/A and sleep. The VLPO nucleus is a group of neurons including W/A-promoting noradrenergic neurons in the locus coeruleus (LC), serotonergic neurons in the dorsal raphe (DR) and histaminergic neurons in the tuberomammillary nucleus (TMN). When OREX is produced and transmitted, the LC, DR and TMN are stimulated, the VLPO is shut off, the extrathalamic pathways are open and the body is W/A. Contrarily, as OREX is reduced, the extrathalamic pathways are blocked, the VLPO turns on, and the body feels tired.

Certain embodiments of this invention focus on increasing OREX impact for energy either by increasing production, decreasing deterioration or by increasing its efficacy. These embodiments include: Calcium Pyruvate, Citicoline Sodium, L-Tyrosine, Caffeine, B Vitamins, and Vitamin C.

In the brain, reduced glucose increases orexin gene expression, orexin concentrations, and increases expression of orexin receptors, thus increasing W/A. OREX uses pyruvate and lactic acid as energy substrates. Pyruvate enhances brain-to-blood glutamate efflux. Glutamate is metabolized to α-ketoglutarate, and in Krebs it is metabolized to oxaloacetate to yield nine (9) ATP molecules. Thus, increased cytosolic pyruvate suppresses orexin cell membrane potential responses to glucose, but still increases ATP. Also, OREX increases the number of N Methyl D Aspartate (NMDAR) receptors, which increases neuronal excitability from glutamate. Certain embodiments of the invention include Calcium Pyruvate.

Increased NMDAR also strongly excites various brain nuclei having important roles in W/A including ACH, dopamine (a catecholamine neurotransmitter and hormone), noradrenalin, and histamine systems. NMDAR activation is also indirectly modulated by ACH through the activation of muscarinic or nicotinic receptors.

OREX promotes cortical activation and attention by increasing cortical ACH release to act on the thalamo-cortical projections. OREX signaling in the BF excites cholinergic neurons and increases prefrontal ACH. As previously described, ACH was the first molecule to be identified as a neurotransmitter important for W/A. Cortical ACH levels are higher during conditions of enhanced or sustained attention. It is also a neuromodulator. Its production is limited by the amount of available Choline. Once absorbed, Citicoline (cytidine 5′-diphosphocholine), a nucleotide composed of cytosine, choline, ribose, and pyrophosphate crosses the blood-brain barrier into the CNS. Certain embodiments of the invention include Citicoline, which provides the brain with choline that readily becomes ACH. Choline is transported into presynaptic neuron cells where it combines with acetyl CoA to synthesize ACH. Calcium is required for sympathetic nerve activity related to W/A. OREX-A also modulates cholinergic neuron activity indirectly by increasing local glutamate release (Krebs-ATP) within the basal forebrain.

OREX signaling in the BF excites dopaminergic neurons. The pituitary gland perceives increased dopamine as an emergency and stimulates the adrenal glands to release adrenaline. L-Tyrosine (L-Tyr) is a precursor in the synthesis of dopamine and noradrenalin. In the CNS, L-Tyr is converted by the enzyme hydroxylase into L-Dopa, the direct precursor of dopamine, which in turn is converted to noradrenalin. Basal forebrain cholinergic neurons project their axons ascending to the medial prefrontal cortex (mPFC) where L-Tyr increases extracellular dopamine and noradrenalin levels in the mPFC. In the PNS, L-Tyr increases extracellular dopamine and noradrenalin levels in the adrenal medulla. Certain embodiments of the invention include L-Tyrosine.

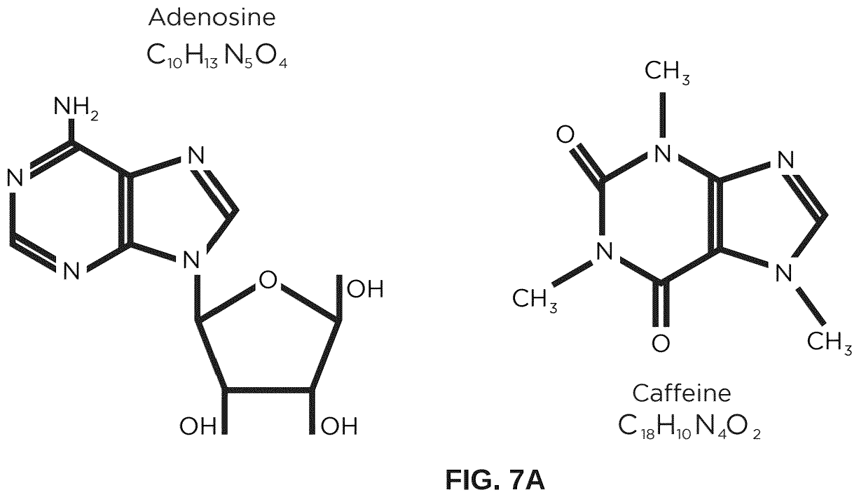

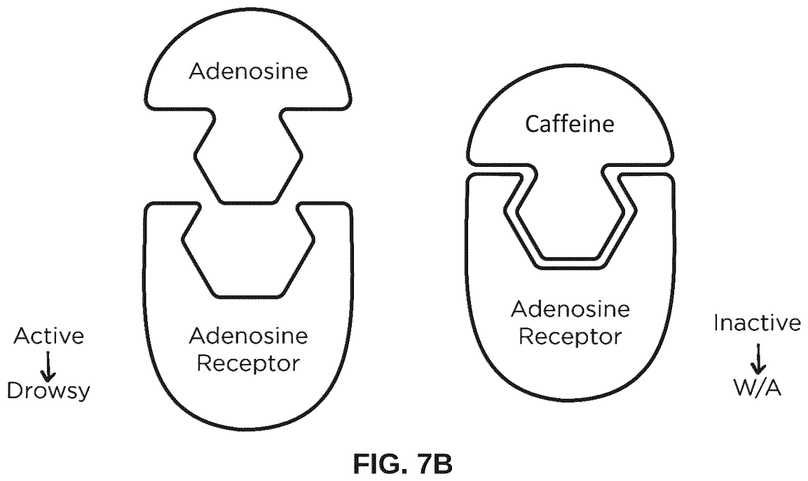

Adenosine (FIG. 7A) is a purine nucleotide and caffeine is a xanthine purine. Less adenosine receptor stimulation leads to more alertness. As ATP is spent, adenosine accumulates. The longer you are awake, the more adenosine you accumulate, which exerts sleep pressure because adenosine slows down cellular activity. When caffeine binds to adenosine receptors in the brain (FIG. 7B), it blocks adenosine from binding. When caffeine binds to adenosine receptors in the brain it does not slow down brain cell activity. In essence, caffeine suppresses adenosine.

Caffeine also increases Orexin release by antagonizing adenosine receptors and relieving adenosine inhibition of orexinergic neurons. By blocking Adenosine 1 receptors, Caffeine promotes W/A by increasing ANS activity through the release of plasma adrenalin and noradrenalin, which stimulate the sympathetic nervous system, playing a role in suppressing hunger, enhancing satiety, and increasing the breakdown of fat cells to be used for energy. Caffeine causes neural excitation in the brain. By blocking Adenosine 2A receptors, Caffeine increases dopamine. Certain embodiments of the invention include Caffeine.

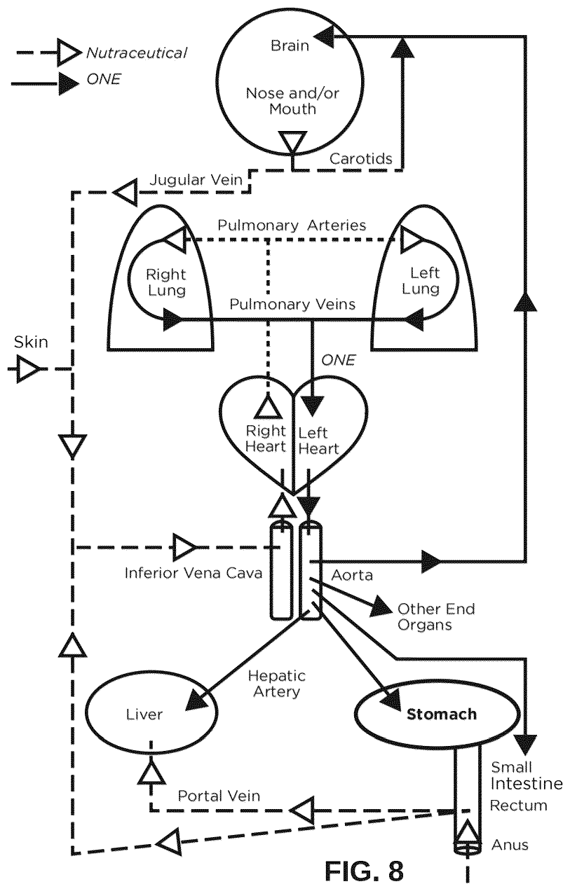

With reference to FIG. 8, certain embodiments of the invention work by direct absorption into the general blood circulation of nutraceuticals via the buccal mucosa, sublingual mucosa, nasal mucosa, rectal and or anal mucosa by gel, strip, film, tablet, caplet, chewable, powder, spray, droplet or auto-injector, suppository and or enema. The extensive vascular plexus of the mouth including but not limited to the floor, beneath the tongue, and buccal areas is drained via the jugular vein. The jugular vein sends the blood via the inferior vena cava to the lungs where it is oxygenated. In the lungs oxygenated nutraceutical-enriched blood (ONE) is carried by the pulmonary veins to the left heart. ONE is transported from the left heart via the aorta and flows through the arterial system to end organs, bypassing the gastrointestinal digestive route. ONE is distributed directly to the brain via arterial branches of the internal and external carotid arteries, passing proximate to, and through, the mouth. As an advantage over the known prior art, ONE travelling directly to the brain impacts the W/A state expediently. In some instances, nutraceuticals are lipidated to promote transport across cell membranes. In some other instances, nutraceuticals are micronized and/or nanonized. Micronization is a process wherein solid particulate matter is milled or ground using modern techniques to reduce the size of a particle to the micrometer range or with nanonization to the nanometer scale (nkamicronization). Micronization improves bioavailability and efficacy of a particle. Micronization enhances the transport of particles across cell membranes. The smaller the particle size, the better the transport. The invention's novel deliveries of nutraceuticals by sublingual or buccal gel, strip, film, tablet, quick dissolving tablet, chewable, powder, spray, droplet or auto-injector, allows the blood to become directly enriched with nutraceuticals for the promotion of W/A, bypassing the gastrointestinal digestive route.

Another embodiment can work by direct absorption in the general blood circulation of nutraceuticals via the nasal mucosa, where the nutraceutical composition may be administered by nasal spray, including, but not limited to, aerosols or pumps sprays, solutions, powders, gels, strips, films, droplets, and/or auto-injectors, and the like. The large surface area of richly vascularized nasal mucosa is drained via the jugular vein. The embodiments can allow for novel deliveries of nutraceuticals that allow the blood to become directly enriched with nutraceuticals, bypassing the gastrointestinal digestive route. Miconization and nanonization of the nutraceuticals potentiate and improve absorption.

In another embodiment of the invention, the very rich portal-caval anastomotic vascular plexus of the rectum and anal mucosa is used for delivery of nutraceuticals by suppository, enema or auto-injector. In the rectum and anus, nutraceuticals are transported by the rectal venous plexus to the inferior vena cava and also to the portal vein via the portal-caval anastomosis, thereby both utilizing and bypassing first pass metabolism of the gastrointestinal digestive route.

Another embodiment of the invention utilizes the skin for dermal absorption delivery of nutraceuticals in the form of cream, lotion, ointment, gel, patch or auto-injector, bypassing the gastrointestinal digestive route. In order to potentiate dermal absorption through the skin, nutraceuticals may be micronized or nanonized to potentiate transport.

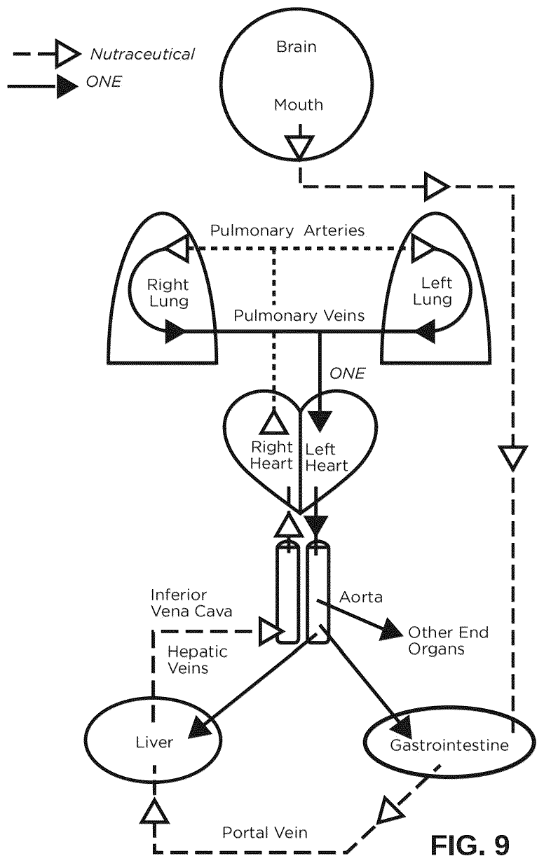

With reference to FIG. 9, another embodiment of the invention utilizes ingestion for the delivery of nutraceuticals in the form of tablets, chewables, quick dissolving tablets, capsules, caplets, gelcaps or liquids. In order to potentiate absorption through the gastrointestine, nutraceuticals may be lipidated, micronized, or nanonized to potentiate transport.

Certain embodiments of the invention use nutraceuticals that promote molecules that act as building blocks and substrates for neuropeptides, hormones, catecholamines and energy including but not limited to: glucose, pyruvate, GABA, glutamate, peptide histidine, isoleucine, corticotrophin releasing hormone, AVP, somatostatin, adrenaline, norepinephrine, epinephrine, noradrenalin, ATP, aspartate, aspartic acid, cysteine, nitric oxide, adenine, riboside, adenosine, ephedrine, pseudoephedrine, N acetyl aspartylglutamate, orexin, serotonin, N methyl D aspartate, albumin, ovalbumin, ovaltransferrin, acetylcholine, ghrelin, leptin, histamine, cortisol, dopamine, L dopamine, cholecystokinin, oxytocin, vasopressin, taurine, arginine, L arginine, L isoleucine, isoleucine, lysine, L lysine, glycine, L glycine, glutamic acid, leucine, beta-hydroxy-beta-methylbutyric acid, grape seed extract, L leucine, L histidine, histidine, L methionine, methionine, alanine, L alanine, beta alanine, L phenylalanine, phenylalanine, serine, L serine, valine, L Valine, L citrulline, caffeine, Camellia sinensis, conephora, betaine, glutamine, L glutamine, glutathione, bacopa monneiri, Indian pennywort, lion's mane, maritime pine bark, proanthocyanidin, huperzine, tyrosine, L tyrosine, Ginkgo biloba, ginseng, choline, creatyl L leucine, alpha GPC, citicoline sodium, citicholine, L citicoline, caffeine, glycogen, all B vitamins (and in all forms), N acetyl L tyrosine, tyrosine, jujube (all forms), chocolate, PEA, phenylethylamine, tryptophan, pilocarpine, physostigmine, nicotine, muscarine, scopolamine, atropine, threonine, L threonine, theanine, L theanine, Proline, Myoinositol, inositol (all forms), calcium, potassium, magnesium (all forms), sodium, phosphorus, chloride, histamine, strawberry, apple cider vinegar, cranberry, acacia, carrot, sour cherry, blackberry, raspberry, acerola, Rubus occidentalis, pomegranate, banana, beet root, flax, mango, papaya, peach, alphalipoic acid, 5HT, xanthine, theophylline, phosphodiesterase, cAMP, Anserine, carnitine, dandelion, valine, zinc, chromium, hardy kiwi, acetyl L carnitine, carnosine SAMe, creatine, bitter orange, Rhodiola rosea, rosavin, salidroside, alpha glycerylphosphatidylcholine, Co enzyme Q 10, ubiquinone, Vitamin D (all forms), Vitamin C (all forms), citric acid, guanidinoacetic acid, apple, guava, pineapple, bromine, orange, sodium citrate, glucuronolactone, sucralose, saccharose, sorbitol, insulin, sugar, pectin, milk thistle, Panax ginseng, American ginseng, bilberry, black crowberry, borage, bottle gourd, cattails, chaparral, chromium pecolinate, cocoplum, forskolin, Coleus forskohlii, common lungwort, cottonwood, dandelion, goldenrod, hawthorn, horsetail, juniper, leeks, oregano, plantain, purslane, sala, skullcap, sugarmaple, thyme, turkey tail, western red cedar, wild ginger, wild rose, wild violets, calendula, glucuronic acid, acacia rigidula, beta methylphenethylamine, DMAA, cytochrome P 450, paraxanthine, trimethylxanthine, chaga, adrafinil, DMEA, acetyl L carnitine, peppermint, rosemary, watermelon, malic acid, water hyssop, grahmi, resveratrol, biotin, phosphatidylcholine, phosphatidylserine, docosahexaenoic acid (DHA), curcumin, piperdine, piperine, capsaicin, cayenne, ginger, cinnamon, quercetin, catechin, epicatechin, chlorogenic acid, onion, manganese, copper, iron, turmeric (all forms), omega 3 fatty acid, fish oil, eicosapentaenoic acid (EPA), spirulina, hovenia dulcis (DHM), N Acetyl cysteine (NAC), cat's claw, devil's club, reishi, moringa, mugwort, peppermint, prickly pear cactus, saffron (all forms), Spanish moss, St. Johns wort, ashwagandha, anise hyssop, gallic acid, phloridzin, procyanidin B, dihydrochalcone, cardamom, clove, cacao, cordyceps, and stinging nettle.

The efficacy of a tested embodiment of the invention is demonstrated by the following example. Capsules were prepared based on a nutraceutical composition of an exemplary embodiment of the invention. Each dose was comprised of two (2) capsules. The dose delivered 732 mg of active ingredients: 25 mg of Vitamin C, 120 mg of Vitamin B1, 3 mg of Vitamin B2, 10 mg of Vitamin B3, 4 mg of Vitamin B6, 500 mcg (microgram, μg) of Vitamin B12, 5 mg of Vitamin B5, 55 mg of Citicoline Sodium, 250 mg of Calcium Pyruvate, 160 mg of Caffeine, and 200 mg of L-Tyrosine.

147 participants were instructed to swallow two (2) capsules each day for five (5) days, and to complete a survey on the sixth day. They were instructed to swallow the capsules no later than five (5) hours before bed.

In the survey, participants were asked to report the following on the days that they swallowed the capsules: (a) whether or not they felt the capsules energized them, (b) whether or not they experienced an energy slump, (c) whether or not they felt the capsules helped them to feel alert, and (d) whether or not they felt the capsules helped them to feel focused.

Of the total 147 participants, 122 (83%) felt some form of positive impact from the capsules. Of the total 147 participants 76 (52%) felt energized, 74 (50%) did not experience energy slumps, 57 (39%) felt more alert, and 56 (38%) felt more focused.

The results of the survey are summarized in Table 1 below.

TABLE 1
Results of all 147 participants.

| | | |
|---|---|---|
| Felt a positive impact | 122 | 83% |
| Felt more energized | 76 | 52% |
| Felt more alert | 57 | 39% |
| Felt more focused | 56 | 38% |
| Didn't experience an energy slump | 74 | 50% |

Table 1 proves that the nutraceutical composition according to the tested embodiment of the invention effectively, on average, improves W/A and thus includes a therapeutically effective amount of each ingredient thereof.

Some variations in formulation are within the scope of these descriptions, such variations are each expected to improve W/A. For example, some embodiments otherwise formulated as the tested embodiment include no directly added caffeine, some such embodiments include no directly added Vitamin C, and some such embodiments can be combined for formulations with neither directly added caffeine nor Vitamin C, as some members of the population prefer caffeine-free supplements, and Vitamin C may be present in other or regular diet items.

Some variations in formulation within the scope of these descriptions are expected to improve W/A generally in keeping with the performance of the tested embodiment of the invention. Such variations include embodiments in which a therapeutically effective amount of each ingredient includes, in a formulation without directly added caffeine and Vitamin C, 100 mg to 120 mg of Vitamin B1, 2 mg to 5 mg of Vitamin B2, 8 mg to 12 mg of Vitamin B3, 4 mg to 6 mg of Vitamin B5, 3 mg to 5 mg of Vitamin B6, 400 μg to 600 μg of Vitamin B12, 50 mg to 60 mg of citicoline sodium, 200 mg to 300 mg of calcium pyruvate, and 150 mg to 250 mg of L-tyrosine. Such variations can further include 150 mg to 1000 mg of caffeine, and/or 20 mg to 3 mg of Vitamin C.

An exemplary therapeutically effective amount of the nutraceutical composition may deliver to the individual, for example, within the range of no less than and no more than the following weight ranges either as or not as micronized and either as or not as lipidated:

| | |
|---|---|
| Caffeine | 15 mg-1 g |
| Calcium Pyruvate | 3 mg-50 g |
| Citicoline Sodium | 10 mg-5 g |
| L-Tyrosine | 15 mg-5 g |
| Vitamin C | 12 mg-3 g |
| Vitamin B1 | 0.04 mg-120 mg |
| Vitamin B2 | 0.25 mg-25 mg |
| Vitamin B3 | 0.9 mg-70 mg |
| Vitamin B5 | 1 mg-30 mg |
| Vitamin B6 | 0.25 mg-40 mg |
| Vitamin B12 | 100 mcg-1 g |

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art.

All embodiments should be regarded as illustrative rather than restrictive. Accordingly, it should be appreciated that variations to those embodiments may be made by those skilled in the art without departing from the scope of the invention as defined by the following claims.

What is claimed is:

1. A nutraceutical composition comprising a therapeutically effective amount of Vitamin B1, Vitamin B2, Vitamin B3, Vitamin B5, Vitamin B6, Vitamin B12, citicoline sodium, calcium pyruvate, and L-tyrosine, wherein said therapeutically effective amount promotes wakefulness/alertness by providing molecular support for glycolysis, Krebs cycle reactions, and electron transport chain (ETC) activity.

2. The nutraceutical composition according to claim 1, wherein the composition comprises 0.04 mg to 120 mg of Vitamin B1, 0.25 mg to 25 mg of Vitamin B2, 0.9 mg to 70 mg of Vitamin B3, 1 mg to 30 mg of Vitamin B5, 0.25 mg to 40 mg of Vitamin B6, 1.5 μg to 1 g of Vitamin B12, 10 mg to 5 g of Citicoline Sodium, 3 mg to 50 g of Calcium Pyruvate, and 15 mg to 5 g of L-Tyrosine.

3. The nutraceutical composition according to claim 1, further comprising a therapeutically effective amount of caffeine, wherein the therapeutically effective amount of caffeine further provides molecular support for wakefulness/alertness by binding to adenosine receptors.

4. The nutraceutical composition according to claim 3, wherein the therapeutically effective amount of caffeine is microencapsulated.

5. The nutraceutical composition according to claim 3, wherein the therapeutically effective amount of caffeine comprises 15 mg to 1 g of caffeine.

6. The nutraceutical composition according to claim 1, further comprising a therapeutically effective amount of Vitamin C, wherein the therapeutically effective amount of caffeine further provides molecular support for wakefulness/alertness by binding to adenosine receptors.

7. The nutraceutical composition according to claim 6, wherein the therapeutically effective amount of Vitamin C comprises 12 mg to 3 g of Vitamin C.

8. The nutraceutical composition according to claim 1, wherein the composition comprises 100 mg to 120 mg of Vitamin B1, 2 mg to 5 mg of Vitamin B2, 8 mg to 12 mg of Vitamin B3, 1 mg to 6 mg of Vitamin B5, 3 mg to 5 mg of Vitamin B6, 400 μg to 600 μg of Vitamin B12, 50 mg to 60 mg of citicoline sodium, 200 mg to 300 mg of calcium pyruvate, and 150 mg to 250 mg of L-tyrosine.

9. A method of promoting wakefulness/alertness in a subject comprising administering to the subject via mucosal absorption in the mouth, nose, respiratory pathway, anus or lower gastrointestinal tract a therapeutically effective amount of the composition according to claim 1.

10. A method of promoting wakefulness/alertness in a subject comprising administering to the subject via dermal absorption a therapeutically effective amount of the composition according to claim 1.

11. A method of promoting wakefulness/alertness in a subject comprising administering to the subject via the gastrointestinal tract through ingestion a therapeutically effective amount of the composition according to claim 1.

12. A method of promoting wakefulness/alertness in a subject comprising administering to the subject via mucosal absorption in the mouth, nose, respiratory pathway, anus or lower gastrointestinal tract a therapeutically effective amount of the composition according to claim 3.

13. A method of promoting wakefulness/alertness in a subject comprising administering to the subject via dermal absorption a therapeutically effective amount of the composition according to claim 3.

14. A method of promoting wakefulness/alertness in a subject comprising administering to the subject via the gastrointestinal tract through ingestion a therapeutically effective amount of the composition according to claim 3.

15. A method of promoting wakefulness/alertness in a subject comprising administering to the subject via mucosal absorption in the mouth, nose, respiratory pathway, anus, or lower gastrointestinal tract, a therapeutically effective amount of the composition according to claim 6.

16. A method of promoting wakefulness/alertness in a subject comprising administering to the subject via dermal absorption a therapeutically effective amount of the composition according to claim 6.

17. A method of promoting wakefulness/alertness in a subject comprising administering to the subject via the gastrointestinal tract through ingestion a therapeutically effective amount of the composition according to claim 6.
